(19) United States
(12) Patent Application Publication
(10) Pub. No.: US 2026/0136142 A1
(43) Pub. Date: May 14, 2026

(54) ACQUISITION SEQUENCES OF BINAURAL HEARING SYSTEMS COMPRISING HEARING IMPLANTS

(71) Applicant: GN Hearing A/S, Ballerup (DK)

(72) Inventor: Michael Løngaa, Farum (DK)

(73) Assignee: GN Hearing A/S, Ballerup (DK)

(21) Appl. No.: 19/312,056

(22) Filed: Aug. 27, 2025

Related U.S. Application Data
(63) Continuation of application No. PCT/EP2024/058120, filed on Mar. 26, 2024.

(30) Foreign Application Priority Data
Mar. 31, 2023 (EP) 23165779.2

(51) Int. Cl. H04R 25/00; A61N 1/05

(52) U.S. Cl. CPC H04R 25/552; A61N 1/0541; H04R 25/554; H04R 2225/67

(57) ABSTRACT

The present disclosure relates to acquisition sequences of binaural hearing systems and corresponding binaural hearing systems. The binaural hearing systems may comprise first and second hearing devices, for example head-wearable devices at a user’s ears, and first and second hearing implants.

### RELATED APPLICATION DATA

This application is a continuation of International Patent Application No. PCT/EP2024/058120 filed on March 26, 2024, which claims priority to, and the benefit of, European patent application No. 23165779.2 filed on March 31, 2023. The entire disclosures of the above applications are expressly incorporated by reference herein.

### FIELD

The present disclosure relates to acquisition sequences of binaural hearing systems and corresponding binaural hearing systems. The binaural hearing systems may comprise first and second hearing devices, for example head-wearable devices at a user’s ears, and first and second hearing implants.

### BACKGROUND

Binaural hearing systems that comprise a pair of hearing implants, such as cochlea hearing implants, may comprise at least four separate devices that are connected via at least three bidirectional wireless communication links during normal operation. A first bidirectional wireless communication link must be connected between a first hearing device, typically arranged at, in or on a user’s ear, and a first hearing implant such as a cochlea implant. A second bidirectional wireless communication link must be connected between a second hearing device, typically arranged at, in or on the user’s opposite ear, and a second hearing implant. Finally, a third bidirectional wireless communication link must be connected between the first and second hearing devices, i.e. a bilateral link, to allow the first and second hearing devices arranged at opposite sides of the user’s head to communicate data, e.g. configured as data packets. The data may for example comprise digital audio signals and/or control information to enable sophisticated binaural processing algorithms and/or synchronize other functions of the first and second hearing devices.

There is need for binaural hearing systems that for example use a common communication protocol between the first and second hearing devices and their respective first and second hearing implants. There is additionally or alternatively a need for binaural hearing systems that exhibit rapid and energy efficient acquisition of each of the first and second bidirectional wireless communication links as well as the bilateral link at start-up of the binaural hearing system. There is additionally or alternatively a need for energy efficient operation of the first and second hearing devices and energy efficient operation of the first and second hearing implants during normal operation of the binaural hearing system, in particular in connection with exchange of data. Such energy efficient operation of the binaural hearing system is advantageous because each of the first and second hearing devices and each of the first and second hearing implants is typically a relatively small device energized by power sources with limited capacity such as rechargeable batteries.

### SUMMARY

A first aspect of the subject disclosure relates to a binaural hearing system comprising:

a first hearing device, a second hearing device, a first hearing implant and a second hearing implant; wherein

the first hearing device is connectable to the first hearing implant via a first bidirectional wireless communication link for exchange of first ipsilateral data packets,

the second hearing device is connectable to the second hearing implant via a second bidirectional wireless communication link for exchange of second ipsilateral data packets,

a bilateral bidirectional wireless communication link is connectable between the first hearing device and second hearing device for exchange of bilateral data packets. The bilateral bidirectional wireless communication link and said first and second bidirectional wireless communication links are preferably configured to operate in accordance with a common communication protocol. The common communication protocol comprises a plurality of consecutive frames each comprising a plurality of time slots and an acquisition sequence. The latter comprises:

acquire the bilateral bidirectional wireless communication link using at least a first processing unit of the first hearing device and a second processing unit of the second hearing device,

acquire the first bidirectional wireless communication link in response to acquisition of the bilateral bidirectional wireless communication link using at least the first processing unit of the first hearing device,

acquire the second bidirectional wireless communication link in response to acquisition of the bilateral bidirectional wireless communication link using at least the second processing unit of the second hearing device.

After successful completion of the acquisition sequence of the binaural hearing system the latter preferably enters a normal operation mode, e.g. “payload mode”, where the first hearing device and the first hearing implant are connected, such as wirelessly connected, through the first bidirectional wireless communication link. The second hearing device and the second hearing implant are likewise connected, such as wirelessly connected, through the second bidirectional wireless communication link during the normal operation after successful completion of the acquisition sequence. Finally, the first hearing device and the second hearing device are connected, such as wirelessly connected, through the bilateral bidirectional wireless communication link during the normal operation after successful completion of the acquisition sequence. The common communication protocol may be proprietary and designed for minimal power consumption since each of the first and second hearing devices and each of the first and second hearing implants typically are relatively small battery powered devices or otherwise energized by a power source with limited capacity.

One of the first hearing device and second hearing device is preferably configured as master and the other one as slave for the purpose of acquiring and operating the bilateral bidirectional wireless communication link using their respective first and second processing units, e.g. master processing unit and slave processing unit, as discussed in additional detail below with reference to the appended drawings.

Each of the first and second hearing devices may comprise a head-wearable housing shaped and sized similarly to a traditional hearing aid for example of so-called BTE, ITE, ITC, CIC or RIC types of housings. The housings may be shaped and sized for placement at, or in, a user’s left or right ear for example shaped and sized for placement behind the user’s left and right ear pinna. The first and second hearing implants may be configured for placement at respective sides of the user’s skull and configured to supply respective nerve stimulus signals to the user’s left and right hearing nerves via implanted electrode arrays.

Each of the first and second bidirectional wireless communication links and the bilateral bidirectional wireless communication link may be based on near-field magnetic coupling, such as NFMI, using respective magnetic coil antennas mounted in the first and second hearing devices. Each of the first and second bidirectional wireless communication links and the bilateral bidirectional wireless communication link may for example use a carrier frequency between 5 MHz and 50 MHz as discussed in additional detail below with reference to the appended drawings.

According to one embodiment of the binaural hearing system at least a subset of the first and second ipsilateral data packets comprises respective digital audio data such as real-time digital audio signals; and/or at least a subset of the bilateral data packets comprises respective digital audio signals or data such as real-time digital audio signals as discussed in additional detail below with reference to the appended drawings.

Each of the first processing unit and second processing unit may comprise a digital signal processor (DSP) and/or a microprocessor such as a software programmable DSP or a software programmable microprocessor. The software programmable DSP or microprocessor may be configured to execute plurality of program instructions configured to implement at least parts of the respective acquisitions of the first and second bidirectional wireless communication links and the bilateral bidirectional wireless communication link in accordance with the common communication protocol. Each of the first processing unit and second processing unit may comprise a dedicated digital state machine configured to handle certain steps of the acquisitions of the first and second bidirectional wireless communication links and the bilateral bidirectional wireless communication link in accordance with the common communication protocol.

According to an embodiment of the common communication protocol and corresponding binaural hearing system, the acquisition of the bilateral bidirectional wireless communication link comprises:

transmit a first synchronization marker from the first processing unit to the second processing unit in a first time slot at the first processing unit, - monitor the plurality of time slots at the second processing unit for the first synchronization marker,

slide the plurality of time slots at the second processing unit with predetermined time steps from frame to frame of the plurality of consecutive frames using a wrap-around scheme,

detect the first synchronization marker at the second processing unit,

transmit an acknowledge message from the second processing unit to the first processing unit in a second time slot at the first processing unit,

synchronize the plurality of time slots at the second processing unit with the plurality of time slots at the first processing unit based on the first synchronization marker,

complete the acquisition of the bilateral bidirectional wireless communication link,

start the exchange of the bilateral data packets through the bilateral bidirectional wireless communication link in the first and second times slots of the consecutive frames.

A “time slot” is the shortest time division of the bilateral bidirectional wireless communication link and first and second bidirectional wireless communication links as defined by the common communication protocol. One data packet may for example be transmitted in one time slot.

A “frame” is a unit of data that comprises a predefined number of time slots as defined by a communication protocol.

“Connectable” shall mean that the wireless communication link in question is configured to establish a wireless connection between specified devices after acquisition of the wireless communication link in question.

A “bidirectional wireless communication link” is a wireless communication link that supports transmission of data packets or data messages from a first device to a second device and transmission of data packets or data messages from the second device to the first device.

“Ipsilateral data packets” are data packets exchanged between a hearing device and a hearing implant arranged at the same side of a user’s head.

A “hearing implant” shall mean that portion of a cochlear implant that is located inside the user’s skull.

According to an embodiment of the common communication protocol and a corresponding binaural hearing system, the acquisition of the first bidirectional wireless communication link comprises:

transmit a second synchronization marker from the first processing unit to a first implant processor of the first hearing implant in a third time slot at the first processing unit,

monitor the plurality of time slots at the first implant processor for receipt of the second synchronization marker,

sliding the plurality of time slots at the first implant processor with predetermined time steps from frame to frame of the consecutive frames using a wrap-around scheme,

detect the second synchronization marker at the first implant processor,

synchronize the plurality of time slots at first implant processor with the plurality of time slots at the first processing unit based on the second synchronization marker,

complete the acquisition of the first bidirectional wireless communication link,

transmit an acknowledge message from the first implant processor to the first processing unit in a fourth time slot at the first processing unit; and

the acquisition of the second bidirectional wireless communication link comprises:

transmit a third synchronization marker from the second processing unit to a second implant processor of the second hearing implant in a third time slot at the second processing unit,

monitor the plurality of time slots, at the second implant processor for receipt of the third synchronization marker,

sliding the plurality of time slots at the second implant processor with predetermined time steps from frame to frame of the plurality of consecutive frames using a wrap-around scheme,

detect the third synchronization marker at the second implant processor,

synchronize the plurality of time slots at second implant processor with the plurality of time slots at the second processing unit based on the third synchronization marker,

transmit an acknowledge message from the second implant processor to the second processing unit in a fourth time slot at the second processing unit,

complete the acquisition of the second bidirectional wireless communication link; and

complete the acquisition sequence and enter the normal operation mode of the binaural hearing system.

Each frame of the plurality of consecutive time frames may comprise at least four time slots such as four overlapping time slots. The length of each of the at least four time slots may be identical as discussed in additional detail below with reference to the appended drawings.

In one embodiment each of the predetermined time steps for the sliding of the plurality of time slots is less than 5 %, for example less than 1 %, of a length of one time slot of the plurality of time slots of the frame as discussed in additional detail below with reference to the appended drawings.

According to an embodiment of the common communication protocol and corresponding binaural hearing system, the first synchronization marker comprises a unique pair ID for pairing the first hearing device and the second hearing device; and

the second synchronization marker comprises a unique pair ID for pairing the first hearing device and first hearing implant; and

the third synchronization marker comprises a unique pair ID for pairing the second hearing device and the second hearing implant.

The first synchronization marker transmitted from the first processing unit to the second processing unit during acquisition of the bilateral bidirectional wireless communication link may for example comprise a first unique pair ID. The second processing unit is configured to:

compare the first unique pair ID of the synchronization marker with a pre-stored unique pair ID,

ignore the first synchronization marker if the first unique pair ID does not match the pre-stored unique pair ID; and

transmit the acknowledge message if the pre-stored unique pair ID matches the first unique pair ID of the first synchronization marker. Certain embodiments of the binaural hearing system utilize three unique pair IDs, i.e. two additional unique pair IDs, such as second and third unique pair IDs, in addition to the first unique pair ID, in the above-mentioned pairings of the first hearing device and the first hearing implant, the first hearing device and the second hearing device and the second hearing device and the second hearing implant. This embodiment is favourable because the use of the respective unique pair IDs of the first, second and third synchronization markers avoids erroneous pairings of devices of the binaural hearing system during the acquisition sequence as discussed in further detail below with reference to the appended drawings.

The synchronization marker may at least comprise:

a predetermined binary pattern,

a time slot indicator for indicating which time slot of the plurality of time slots at the first processing unit that holds the synchronization marker, and optionally and error-detecting code.

The common communication protocol may comprise a plurality of super frames each comprising a plurality of individual frames, such as between 4 and 32 individual frames, of the plurality of consecutive frames. In the latter embodiment, the second processing unit is preferably configured to determine an offset in frames between consecutive super frames,

transmit the offset to the first processing unit via the bilateral bidirectional wireless communication link while the first processing unit is configured to read the offset and adjust timing of the super frames at the first processing unit to align super frames at the first and second processing units.

According to one embodiment of the binaural hearing system, the acquisition sequence comprises:

maintain connection through the bilateral bidirectional wireless communication link during the acquisition of the first bidirectional wireless communication link and during the acquisition of the second bidirectional wireless communication link.

A second aspect of the subject disclosure relates to an acquisition sequence, e.g. a computer-implemented acquisition method, in accordance with the common communication protocol. The acquisition sequence may be carried out by a first processing unit and a second processing unit of the first hearing device and the second hearing device, respectively, operating in a concurrent and coordinated manner to acquire a bilateral bidirectional wireless communication link. The acquisition sequence is further configured for acquiring respective connections between a first hearing device and a first hearing implant, a second hearing device and a second hearing implant and between the first hearing device and the second hearing device. The acquisition sequence comprising:

acquire a bilateral bidirectional wireless communication link between the first hearing device and the second hearing device using at least a first processing unit of the first hearing device and a second processing unit of the second hearing device,

exchange bilateral data packets between the first hearing device and the second hearing device,

acquire a first bidirectional wireless communication link between the first hearing device and the first hearing implant, in response to acquisition of the bilateral bidirectional wireless communication link, using at least the first processing unit of the first hearing device,

exchange first ipsilateral data packets between the first hearing device and the first hearing implant,

acquire a second bidirectional wireless communication link between the second hearing device and second hearing implant, in response to acquisition of the bilateral bidirectional wireless communication link, using at least the second processing unit of the second hearing device,

exchange second ipsilateral data packets between the second hearing device and the second hearing implant

enter normal operation of the binaural hearing system.

One embodiment of the acquisition sequence comprises:

exchange the bilateral data packets in first and second time slots at the first processing unit of respective frames of the plurality of consecutive frames,

exchange the first ipsilateral data packets in third and fourth time slots at the first processing unit of the respective frames of the plurality of consecutive frames,

exchange the second ipsilateral data packets using in the third and fourth time slots at the second processing unit of respective frames of the plurality of consecutive frames.

### BRIEF DESCRIPTION OF THE DRAWINGS

In the following exemplary embodiments are described in more detail with reference to the appended drawings, wherein:

FIG. 1 schematically illustrates an exemplary binaural hearing system mounted on a user’s head in accordance with some embodiments,

FIG. 2 shows respective schematic block diagrams of first and second hearing devices and associated first and second hearing implants of the exemplary binaural hearing system,

FIG. 3 schematically illustrates transmission of a first synchronization marker from the first hearing device to the second hearing device during acquisition of a bilateral bidirectional wireless communication link according to embodiments of the exemplary binaural hearing system,

FIG. 4 schematically illustrates exchange of bilateral data packets between the first hearing device and the second hearing device after the acquisition of the bilateral bidirectional wireless communication link according to embodiments of the exemplary binaural hearing system,

FIG. 5 schematically illustrates acquisition of the first bidirectional wireless communication link and acquisition of the second bidirectional wireless communication link in accordance with embodiments of the exemplary binaural hearing system,

FIG. 6 schematically illustrates exchange of first and second ipsilateral data packets and exchange of bilateral data packets during normal operation, i.e. payload mode, of the binaural hearing system after completion of an acquisition sequence in accordance with embodiments of the exemplary binaural hearing system,

FIG. 7 illustrates features of an exemplary synchronization marker utilized during the acquisition of the bilateral bidirectional wireless communication link in accordance with embodiments of the exemplary binaural hearing system,

FIG. 8 illustrates exemplary super-frame structures each comprising a plurality of frames of the ipsilateral data packets and bilateral data packets in accordance with embodiments of the exemplary binaural hearing system,

FIG. 9 and FIG. 10 are flow charts illustrating respective processing steps carried out by the first and second processing units of the first and second hearing devices, respectively, during acquisition of the bilateral bidirectional wireless communication link according to embodiments of the exemplary binaural hearing system.

### DETAILED DESCRIPTION

Various embodiments are described hereinafter with reference to the figures. Like reference numerals refer to like elements throughout. Like elements will, thus, not be described in detail with respect to the description of each figure. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

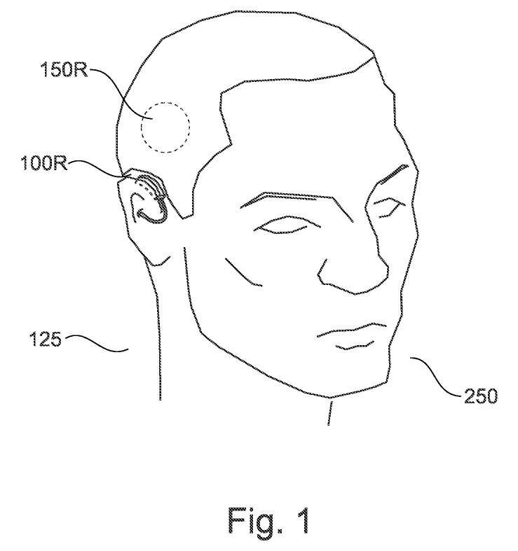

FIG. 1 schematically illustrates an exemplary binaural hearing system 125 mounted on a user’s head 250. The exemplary binaural hearing system 125 comprises a second hearing device 100R and a second hearing implant 150R for example mounted at a right side of the user’s head 250. The binaural hearing system 125 further comprises a first hearing device and a first hearing implant (not shown) that are both arranged at the opposite side of the user’s head 250 and therefore not visible on the drawing. The second hearing implant 150R and the first hearing implant are adapted for surgical implantation in opposite sides of the user’s skull and are therefore invisible from the outside when implanted.

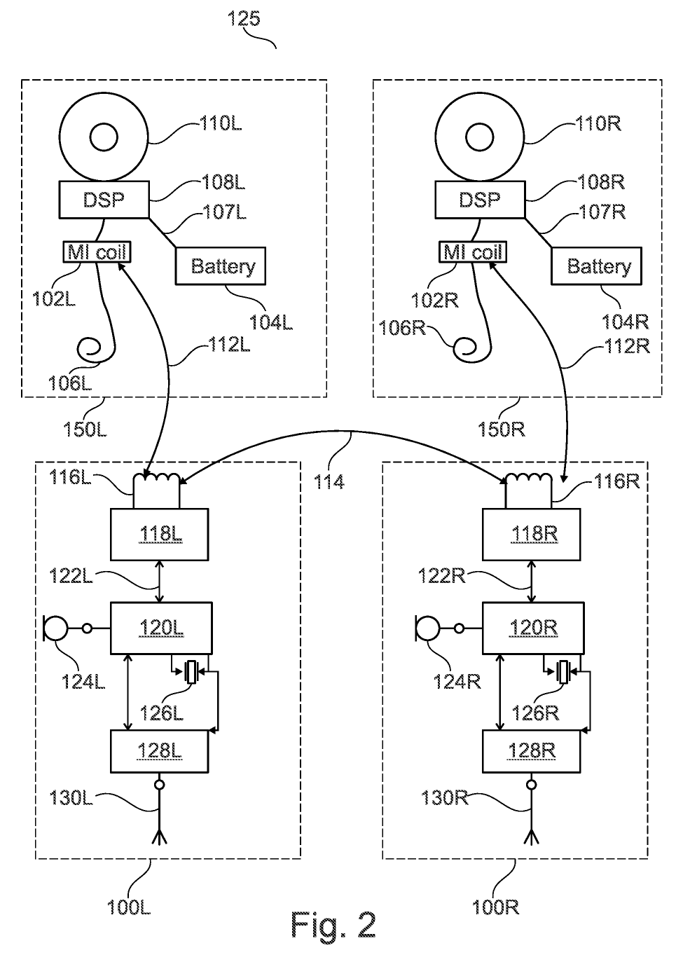

FIG. 2 is a block diagram of the binaural hearing system 125. The first hearing device 100L is connectable to the first hearing implant 150L via a first bidirectional wireless communication link 112L (“first link”) for exchange of first ipsilateral data packets. The second hearing device 100R is likewise connectable to the second hearing implant 150R via a second bidirectional wireless communication link 112R (“second link”) for exchange of second ipsilateral data packets.

The binaural hearing system 125 further comprises a bilateral bidirectional wireless communication link 114 (“bilateral link”, or “third link”) that is connectable between the first hearing device 100L and second hearing device 100R for exchange of bilateral data packets 430, 480 illustrated on FIG. 4. The first hearing device 100L and second hearing device 100R are preferably configured to use respective first and second magnetic coil antennas 116L, 116R for wireless data communication with the first hearing implant 150L and second hearing implant 150R, respectively. The bilateral link 114, the first link 112L and the second link 112R are preferably configured to utilize, i.e. operate in accordance with, a common data communication protocol. The common data communication protocol preferably comprises a plurality of consecutive time frames that each comprises a plurality of time slots holding the first and second ipsilateral data packets 455, 460, 405, 410, respectively, and the bilateral data packets 430, 480 as illustrated on FIG. 6. The respective pluralities of first and second ipsilateral data packets 455, 460, 405, 410, respectively, and bilateral data packets 430, 480 are structured, transmitted and received, e.g. exchanged, in accordance with the common data communication protocol as discussed in further detail below.

The skilled person will understand that certain types of data packets such as synchronization markers and acknowledge messages may be wirelessly exchanged, e.g. transmitted, between the first hearing device 100L and second hearing device 100R, between the first hearing device 100L and the first hearing implant 150L and between the second hearing device 100R second hearing implant 150R before or during the respective acquisitions of the bilateral link 114, first link 112L and second link 112R.

The bilateral data packets exchanged by the bilateral link 114 may comprise digital audio signals or samples and control information transmitted by respective ones of the first and second hearing devices 100L, 100R. Each of the first and second hearing devices 100L, 100R may transmit the digital audio samples and control information or control data to the first and second hearing implants 150L, 150R, respectively. Each of the first and second hearing implants 150L, 150R, preferably utilizes the first and second control units 108L, 108R, respectively, e.g. first and second implant processors 108L, 108R and may additionally be configured to transmit the respective control data to the first and second hearing devices 100L, 100R via the first link 112L and second link 112R. In the latter embodiment both of the first link 112L and second link 112R are adapted for bidirectional data transfer.

Each of the first link 112L, second link 112R and bilateral link 114 may in certain embodiments be based on, or comprise, near-field magnetic coupling, such as NFMI links. The first link 112L may accordingly comprise a first magnetic coil antenna 116L of the first hearing device 100L and a first magnetic coil antenna 102L of the first hearing implant 150L. The second link 112R may likewise comprise a second magnetic coil antenna 116R of the second hearing device 100R and a second magnetic coil antenna 102R of the second hearing implant 100L. Each of first link 112L, second link 112R and bilateral link 114 may for example utilize a carrier frequency between 5 MHz and 50 MHz, such as between 9 MHz and 27 MHz for wireless transmissions of the first and second ipsilateral data packets 455, 460, 405, 410, respectively, and the bilateral data packets 430, 480. The first hearing implant 150L comprises the first control unit 108L that is connected to the first magnetic coil antenna 102L of the first hearing implant 150L for receipt and processing of the first ipsilateral data packets transmitted by the first hearing device 100L. The first control unit 108L is further configured to transmit the first ipsilateral data packets to the first hearing device 100L via the first link 112L. In some embodiments, the first control unit 108L of the first hearing implant 150L may comprise a digital signal processor (DSP) and/or a microprocessor. Certain embodiments of the first hearing implant 150L may comprise a first rechargeable battery assembly 104L. The first rechargeable battery assembly 104L is configured for receiving electrical power from a first receiver (Rx) charging coil 110L during dedicated recharging operations or sessions. The first receiver (Rx) charging coil 110L may be energized by an appropriately positioned external, i.e. outside the user’s skull, transmitter (Tx) charging coil (not shown) of a charging device (not shown) during the dedicated recharging sessions. This allows wireless transfer of power to the first rechargeable battery assembly 104L. The first rechargeable battery assembly 104L is preferably coupled to a power supply input of the first control unit 108L to energize the latter as schematically indicated by a first power line or wire 107L. The control unit 108L may comprise a microprocessor for example software programmable microprocessor.

The first hearing implant 150L further comprises a first electrode array 106L for insertion into a cochlea of the user by implantation. The first electrode array 106L may be electrically coupled to the first control unit 108L that supplies suitable electrode stimuli signals to the first electrode array 106L to stimulate the user’s cochlea nerve. The skilled person will understand that these electrode stimuli signals may be generated by the first control unit 108L and derived by the latter based on the first ipsilateral data packets, in particular digital audio signals or data embedded or held in the first ipsilateral data packets. The skilled person will appreciate that corresponding functions, structures and features of the second hearing implant 150R such as a second control unit 108R, a second electrode array 116R etc. may be largely identical or similar to those of the first hearing implant 150L as indicated by corresponding reference numerals.

The block diagram on FIG. 2 further illustrates an exemplary embodiment of the first hearing device 100L. The first hearing device 100L comprises the first magnetic coil antenna 116L which is electrically connected to a first transceiver 118L. The first transceiver 118L is configured to repeatedly switch between Tx and Rx modes to modulate and demodulate incoming and outgoing data of the first ipsilateral data packets and bilateral data packets 430, 480. The first transceiver 118L may be configured to convert the first ipsilateral data packets and bilateral data packets 430, 480 to a format understood by a first processing unit 120L of the first hearing device 100L. The first transceiver 118L may be electrically connected to the first processing unit 120L e.g. through a first data line or first data bus 122L for transmission of the first ipsilateral data packets and the bilateral data packets to the first processing unit 120L.

In some embodiments, the first processing unit 120L may comprise a digital signal processor (DSP) and/or a microprocessor such as a software programmable DSP or a microprocessor.

The first hearing device 100L further comprises one or more first microphones 124L that are coupled to an appropriate audio interface of the first processing unit 120L. The first processing unit 120L may be configured to execute a suitable operating system. The operating system may be configured to manage various hardware and software resources of the first hearing device 100L such as handling of the common communication protocol, computation of monaurally or bilaterally beamformed microphone signals, hearing loss compensation processing of the microphone signal(s), the first wireless data communication interface 118L, e.g. comprising the first transceiver 118L, certain memory resources etc. The operating system may schedule tasks for efficient use of hearing device resources and may further include accounting software for cost allocation, including power consumption, processor time, memory locations, wireless transmissions, and other resources. The operating system may be stored in and retrieved from a non-volatile memory (not shown), e.g. flash memory or EEPROM, of the first processing unit 120L.

Each of the first and second hearing devices 100L,100R may comprise a housing of a type that is well-known from the hearing aid industry like so-called BTE, ITE, ITC, CIC or RIC housing types. These housing types are shaped and sized for placement at, or in, the user’s ear. The first hearing device 100L further comprises a first system clock generator 126L that is configured to supply system clock signals to various digital logic circuits and components of the first hearing device 100L including the first processing unit 120L as schematically illustrated. A nominal value of a clock frequency of the first system clock generator 126L may lie between 2 MHz and 64 MHz such as between 10 MHz and 50 MHz. The first processing unit 120L may be configured to derive respective lengths of certain time slots and time frames of the common communication protocol of the first link 112L and the bilateral link 114 as discussed in additional detail below.

The first hearing device 100L may additionally comprises a second, optional, wireless communication interface, such as a first radio interface 128L, and a first RF antenna 130L configured to jointly communicate through a second wireless communication interface, such as a first radio link (not shown). The first radio link and radio interface 128L may be configured to operate in the 2.4 GHz industrial scientific medical (ISM) band. The first radio link and radio interface 128L may be compliant with a Bluetooth standard, such as Bluetooth LE. This first radio interface 128L may provide convenient data connectivity to various types of portable communication devices like smartphones, mobile phones, tablets and personal computers etc. due to the industry standard compatible nature of the first radio link. Various types of control data and digital audio signals may be transmitted from the portable communication device to the first hearing device 100L and vice versa. The second hearing device 100R may for the same purposes comprise a similar optional second wireless communication interface, such as a second radio interface 128R and second RF antenna 130R as illustrated. In the following disclosure the first hearing device 100L of the binaural hearing system 125 is assigned as a master device during execution of the common communication protocol while the second hearing device 100R is configured as a slave device. The skilled person will understand that the system clock signal of the first, e.g. master, processing unit 120L may control timing of the wireless transmissions of respective data packets through the first link 112L, second link 112R and bilateral link 114 at least after the acquisitions of the first link 112L, second link 112R and bilateral link 114 where the acquisition sequence is completed and normal operation of the binaural hearing system 125 commenced.

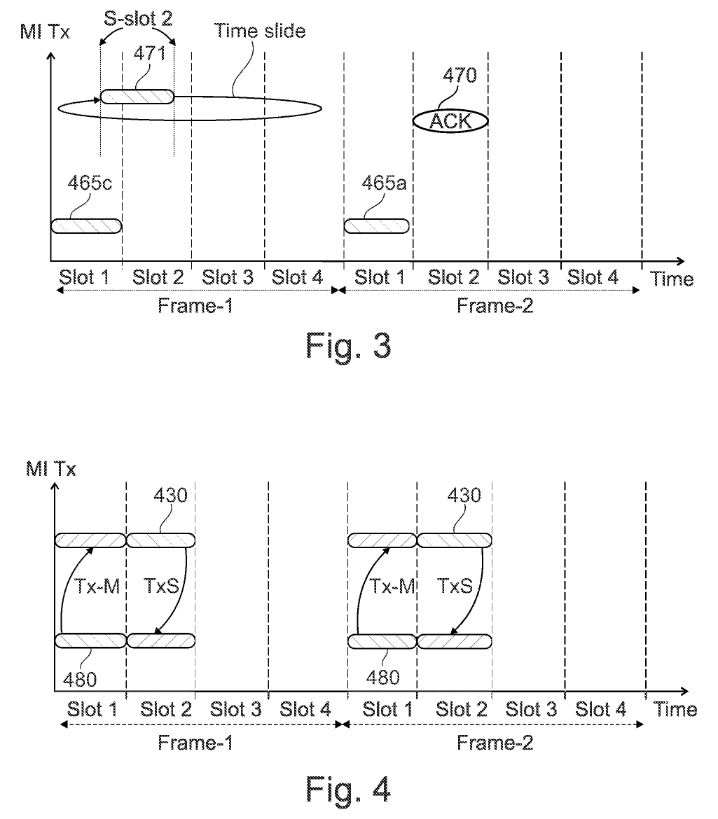

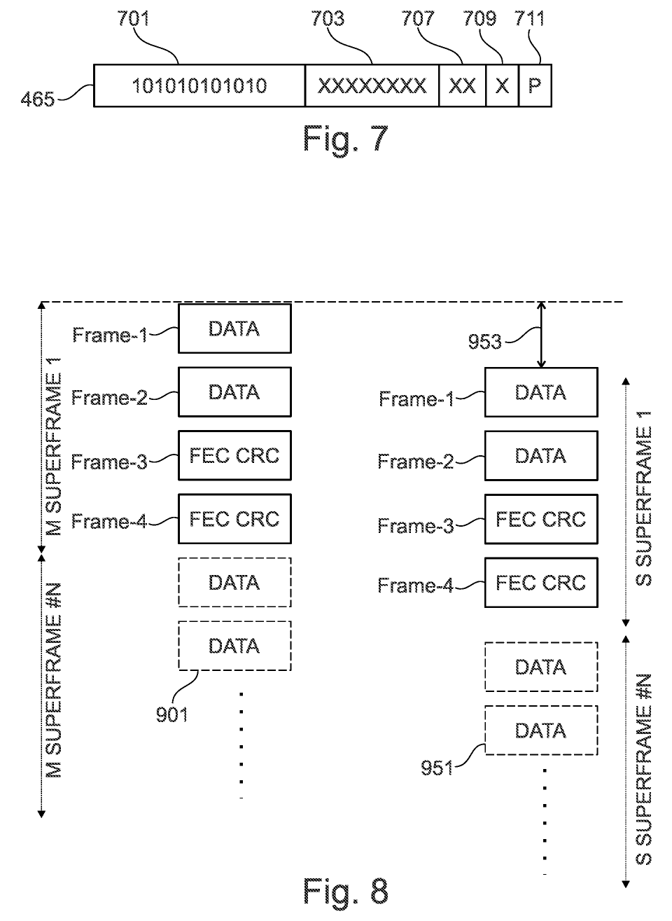

FIG. 3 schematically illustrates transmission of a first synchronization marker 465c from the first, or master, processing unit 120L of the first hearing device 100L to the second or slave hearing device 100R during acquisition of the bilateral link 114 according to embodiments of the common communication protocol of the exemplary binaural hearing system 125. During the acquisition of the bilateral link 114, the master processing unit 120L operates as a master device towards the second hearing device 100R or vice versa. The master processing unit 120L may start to repeatedly transmit the first synchronization marker 465c to the second hearing device 100R using one of a plurality of time slots, e.g. time slots 1-4, of each frame, Frame-1, Frame-2 etc., of a plurality of consecutive frames. The indicated time slots 1-4 are referenced to the master processing unit 120L. Time slot 1, at the master processing unit 120L, is selected for transmission of the first synchronization marker 465c in the present example as illustrated and made in accordance with the common communication protocol. The second hearing device 100R resides concurrently in a receipt mode such that the second, or slave, processing unit 120R of the second hearing device 100R monitors the bilateral link 114 to detect receipt of the first synchronization marker 465c. However, the respective time slots 1-4 at the master processing unit 120L and the slave processing unit 120R are most likely more or less misaligned or asynchronous at the current acquisition step of the acquisition sequence. This misalignment of the respective time slots 1-4 at the master processing unit 120L and the slave processing unit 120R is caused by free-running clock generators and system clock signals at the master processing unit 120L and the slave processing unit 120R. As illustrated by FIG. 3 where the second hearing device 100R, e.g. the second processing unit 120R, searches for the first synchronization marker 465c in an exemplary time slot 471 at the second hearing device 100R which exemplary time slot 471 is shifted or misaligned, as indicated by the S-slot-2 arrow, with about one-fourth of the length of one time slot relative to the time slots at the master processing unit 120L. This misalignment of time slots prevents the second hearing device 100R from detecting the first synchronization marker 465c because the latter extends across two adjacent time slots at the second hearing device 100R. The slave processing unit 120R advances or slides its time slots 1-4, such as the exemplary time slot 471, with predetermined time steps between frames of the plurality of consecutive frames using a wrap-around scheme as schematically indicated by “Time slide” arrow. The wrap-around scheme means that that the sliding of the time slots at the slave processing unit 120R, such as S-slot 2, jumps back to time slot 1 when the end of time slot 4 is reached by the sliding operation. The predetermined time step for the slot sliding is preferably much shorter than a length of one time slot such as less 5 % or less than 1 % of the length of one time slot to gradually align the time slots 1-4 at the slave processing unit 120R with the time slots 1-4 at the master processing unit 120L. The slave processing unit 120R may continue to slide its time slots 1-4 by a wrap-around scheme until the latter time slots are sufficiently aligned with the time slots 1-4 at the master processing unit 120L to detect receipt of the first synchronization marker 465c at the slave processing unit 120R. The slave processing unit 120R may check the unique pair ID (field 703 FIG. 7) of the received first synchronization marker 465c to ensure it is transmitted from the paired device, i.e. the first hearing device 100L in the present situation. The search for the first synchronization marker 465c by the slave processing unit 120R may optionally comprise recognition of a predetermined search sequence 701 of the first synchronization marker 465c (FIG. 7) as described in further detail below.

The slave processing unit 120R may be configured to ignore the first synchronization marker 465c if it is not transmitted by the paired device, i.e. the unique pair ID of the received synchronization marker does not match that of the first synchronization marker 465c. In the latter situation slave processing unit 120R may thereafter continue the search for the first synchronization marker 465c. When the unique pair ID of the received synchronization marker is a match to that of the first synchronization marker 465c, the slave processing unit 120R may respond by detecting the time slot, e.g. time slot 1, at the master processing unit 120L in which the first synchronization marker 465c was transmitted. The master processing unit 120L may be configured to the time slot of the transmission of the first synchronization marker 465c by reading a slot indicator bit or field of the first synchronization marker as discussed in further detail below. The slave processing unit 120R may, based on that time slot identification, align, i.e. synchronize, the time slots at the slave processing unit 120R such as S-slot 2 with the corresponding time slots at the master processing unit 120L of the first hearing device 100L. Hence, synchronize data packet exchange or communication between the slave processing unit 120R and the master processing unit 120L through the bilateral link 114.

The slave processing unit 120R is thereafter configured to shift to a transmission mode and transmit an acknowledge message 470 (ACK) to the master processing unit 120L in time slot 2 at the master processing unit 120L as schematically illustrated by FIG. 3. Thereafter, the master processing unit 120L may respond by completing or terminating the acquisition of the bilateral link 114 because the latter is now appropriately connected between the master processing unit 120L and the slave processing unit 120R in accordance with the exemplary embodiments of the common communication protocol of the binaural hearing system 125. The master processing unit 120L and the slave processing unit 120R are accordingly ready to exchange the bilateral data packets 430, 480 using time slots 1 and 2 of the plurality of consecutive frames as illustrated by FIG. 4. In time slot 1 of frame 1 the master processing unit 120L transmits a first bilateral data packet 480 to the slave processing unit 120R which resides in a receipt mode or state during time slot 1. In time slot 2 of frame 1, the slave processing unit 120R transmits a second bilateral data packet 430 to the master processing unit 120L which has switched to a receipt mode or state during time slot 2.

The skilled person will understand that other embodiments of the common communication protocol may specify more than four time slots per frame such as between 5 and 16 time slots. Irrespective of the actual number of time slots, such as at least four time slots, these are preferably non-overlapping time slots. Each time slot may have a length between 24 µs and 384 µs for example depending a number of bits of at least some of the exchanged data packets. The length of each of the time slots may be identical. Each of the first ipsilateral data packets 455, 460 and the second ipsilateral data packets 405, 410 may comprise identical number of bits for example between 4 bits and 96 bits such as between 6 bits and 24 bits. The number of bits in a particular data packet may depend on content type, i.e. digital audio signals, control information or a combination of both.

At least some of the first and second bilateral data packets 480, 430 comprise digital audio signals and/or control information. The control information may be held in a packet header and the digital audio signals held in a packet payload. The digital audio signals may be perceptually encoded to reduce the amount of audio data in the messages for transmission. The respective digital audio signals of the first and second bilateral data packets 480, 430 may be generated by, or derived, from respective microphone arrangements of the master and slave hearing devices 100L,100R such as the one or more first microphones 124L and one or more second microphones 124R (FIG. 2). These microphone arrangements 124L, 124R may for example be integrated in the respective housings of the first/master and second/slave hearing devices 100L, 100R, respectively. The respective digital audio signals of the bilateral data packets 430, 480 may alternatively be derived from remote microphone arrangements (not shown) wirelessly coupled to the first/master and/or second/slave hearing devices 100L, 100R. The sound may comprise speech, noise or any mixture thereof for example sound present in a user’s external environment or sound streamed sound from an audio enabled portable device.

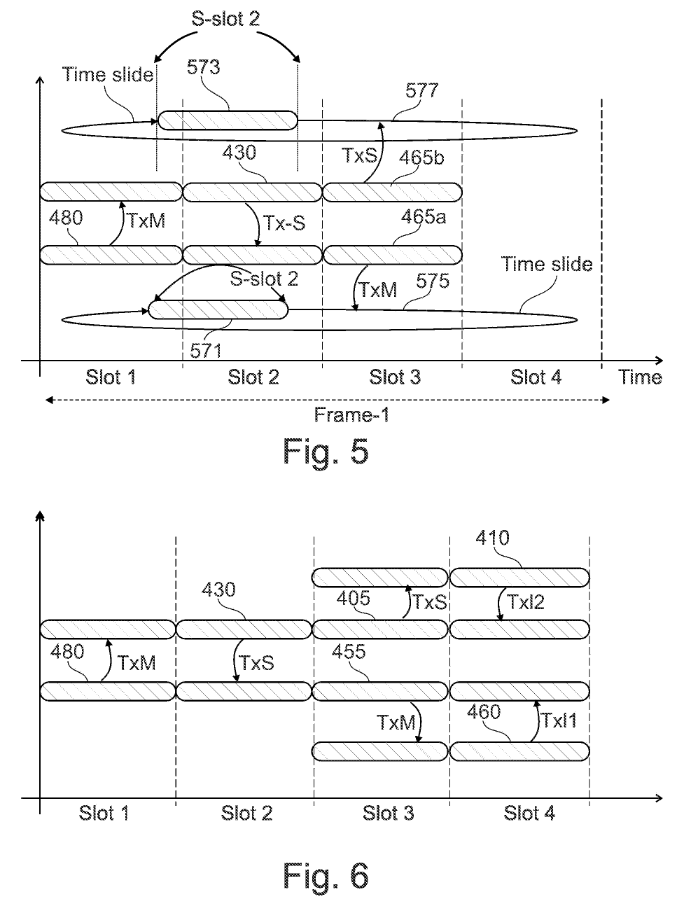

FIG. 5 schematically illustrates acquisition of the first link 112L and acquisition of the second link 112R in accordance with embodiments of the exemplary binaural hearing system 125. As outlined above the acquisition of the bilateral link 114 has been completed and in response the master processing unit 120L initiates the acquisition of the first link 112L and the acquisition of the second link 112R. FIG. 5 illustrates how the master processing unit 120L and the slave processing unit 120R exchange the first and second bilateral data packets 480, 430 using time slots 1 and 2, respectively, of a frame. The master processing unit 120L may further be configured to repeatedly generate and transmit a second synchronization marker 465a to the first control unit 108L of the first hearing implant 150L using an unoccupied time slot of the frames, like time slot 3, at the master processing unit 120L. The first control unit 108L, and consequently the first hearing implant 150L as well, operates in its receipt mode and searches for the second synchronization marker 465a in a time slot 571 at the first control unit 108L. The exemplary time slot 571 is shifted or misaligned, as indicated by the S-slot-2 arrow, with about one-fourth of the length of one time slot relative to the time slots at the master processing unit 120L which are indicated on the x-axis. This misalignment of time slots prevents the first control unit 108L from detecting the second synchronization marker 465a for the reasons discussed above in connection with the first synchronization marker 465c. The first control unit 108L therefore advances or slides its time slots 1-4, such as the exemplary time slot 571, with predetermined time steps from frame to frame of the plurality of consecutive frames using a wrap-around scheme as schematically indicated by a first “Time slide” arrow 575. The predetermined time step is preferably shorter than the length of one time slot such as less than 5 % or less than 1 % of the length of one time slot. The predetermined time step may be 250 ns for a slot length of about 48 µs. Following this scheme, the first control unit 108L is configured to gradually align the time slots 1-4 at the first control unit 108L, i.e. at the first hearing implant 150L, with the time slots 1-4 at the master processing unit 120L. The first control unit 108L may continue to slide its time slots 1-4 until the latter time slots are sufficiently aligned with the time slots 1-4 at the master processing unit 120L to detect receipt of the second synchronization marker 465a at the first control unit 108L. The first control unit 108 may optionally proceed to check the unique pair ID of the received second synchronization marker 465a to ensure, such as test whether it is transmitted from a paired device, i.e. the first hearing device 100L in the present situation. The first control unit 108L may be configured to ignore the received second synchronization marker 465a if it is not transmitted by the paired device, i.e. the unique pair ID of the received synchronization marker does not match that of the second synchronization marker 465a. In the latter situation, the first control unit 108L may thereafter continue searching for the second synchronization marker 465a. When the unique pair ID of the received second synchronization marker 465a is a match, the first control unit 108L may respond by reading the slot indicator 709 of the second synchronization marker 465a and align its time slots 1-4 at with the corresponding time slots at the master processing unit 120L to synchronize the ipsilateral data packet exchange between the first hearing implant 150L and the first hearing device 100L through the first link 112L. The first control unit 108L thereafter shifts to a transmission mode and transmit an acknowledge message (not shown) to the master processing unit 120L in the unoccupied time slot 4 at the master processing unit 120L.

The master processing unit 120L may respond to receipt of the acknowledge message transmitted by the first control unit 108L of the first hearing implant 150L by completing the acquisition of the first link 112L because the latter is now appropriately connected between the first hearing device 100L and the first hearing implant 150L in accordance with the exemplary embodiments of the common communication protocol of the binaural hearing system 125. The first hearing device 100L and the first implant 150L are accordingly ready to exchange the first ipsilateral data packets 455, 460 using time slots 3 and 4 of the plurality of consecutive frames as illustrated by FIG. 6. In time slot 3 of frames 1, the first hearing device 100L transmits, TxM, the first ipsilateral data packet 455 to the first hearing implant 150L.

The first hearing implant 150L resides in the receipt mode or state in time slot 3. In time slot 4 of frame 1, the first hearing implant 150L transmits, TxI1, the second ipsilateral data packet 460 to the master processing unit 120L which now has switched to a receipt mode or state, from the previous transmit mode in time slot 3. Further, in time slot 4 of the frames, the second hearing implant 150R transmits, TxI2, a second ipsilateral data packet 410 to the slave processing unit 120R. The slave processing unit 120R has switched to a transmission mode or state from its previous receipt mode in time slot 3. The skilled person will appreciate that the acquisition of the second link 112R of the exemplary binaural hearing system 125 may follow a corresponding acquisition scheme to that of the first link 112L to create the connection, such as wireless connection, between the second hearing device 100R and the second hearing implant 150R. Preferably, the second or slave processing unit 120R operates as a master device towards the second hearing implant 150R, i.e. towards the second control unit 108R, during the acquisition of the second link 112R. Briefly, the second processing unit 120R may be configured to repeatedly generate and transmit the third synchronization marker 465b to the second control unit 108R of the second hearing implant 150R using an unoccupied time slot of the frames, like time slot 3, at the master processing unit 120L. The third synchronization marker 465b has preferably a different unique pair ID, than that of each of the first and second synchronization markers 465c, 465a, respectively. The third synchronization marker 465bis preferably utilized by the second processing unit 120R and the second control unit 108R of the second hearing implant 150R for the acquisition of the second link 112R. The second control unit 108R advances or slides, as indicated by a second “Time slide” arrow 577, its time slots 1-4, such as the exemplary time slot 573, with predetermined time steps in the same manner as described in detail above in connection with the acquisition of the first link 112L. After completions of the acquisitions of the first and second links 112L, 112R, the master processing unit 120L responds by completing, e.g. terminating, the acquisition sequence of the exemplary binaural hearing system 125. The master processing unit 120L, the slave processing unit 120R, the first hearing implant 150L and second hearing implant 120R enter normal operation, i.e. a “payload mode”, where the first and second ipsilateral data packets 455, 460, 405, 410, respectively, are exchanged in time slots 3 and 4 and the bilateral data packets 480, 430 are exchanged in time slots 1 and 2, respectively, of the consecutive frames as schematically illustrated on FIG. 6.

Some embodiments of the exemplary binaural hearing system 125 and common communication protocol comprise that the master processing unit 120L is configured to respond to receipt of each of the acknowledge messages (470 on FIG. 4) by starting a count-down sequence before each link acquisition is completed. The count-down sequence may comprise an exchange of several rounds, e.g. 2, 3 or 4 rounds, of the synchronization marker in question and the accompanying acknowledge message before termination of the link acquisition. The optional use of count-down sequences may further verify the reliability of the wireless link in question.

The acquisition of the first link 112L may establish the connection between the first processing unit 120L and the first hearing implant 150L by aligning, e.g. synchronizing, the plurality of time slots at the first processing unit 120L with the corresponding time slots at the first hearing implant 150L. Likewise, the acquisition of the second link 112R may establish the connection between the second processing unit 120R and the second hearing implant 150R by aligning, e.g. synchronizing, the plurality of time slots at the second processing unit 120R with the corresponding time slots at the second hearing implant 150R. The acquisition of the bilateral link 114 may establish the connection between the first and second processing unit 120L, 120R, and hence between the first and second hearing devices 100L, 100R, by aligning, e.g. synchronizing, the plurality of time slots at the first processing unit 120L with the corresponding time slots at the second processing unit 120R. Consequently, after completion of the acquisition sequence and entry into normal operation of the binaural hearing system 125, the first processing unit 120L, the first hearing implant 150L, the second processing unit 120R and the second hearing implant 150R are configured for synchronous exchange of their respective data packets. This feature inter alia reduces power consumption and/or supports robust real-time transmission of the digital audio signals of the binaural hearing system 125.

The time-slot based scheme for synchronous transmission of the first and second ipsilateral data packets 455, 460, 405, 410, respectively, and first and second bilateral data packets 480, 430 during normal operation of the exemplary binaural hearing system 125, as devised by the common communication protocol, leads to efficient use of the respective bandwidths of the wireless links 112L, 112R and 114. This time-slot based scheme for synchronous transmission of the respective data packets during normal operation of the exemplary binaural hearing system 125 is additionally energy efficient for the respective transceiver circuitries, such as respective transceivers 118L, 118R, of the master processing unit 120L, the slave processing unit 120R and respective transceivers of the first hearing implant 150L and second hearing implant 150R.

FIG. 7 illustrates layout of an exemplary synchronization marker 465 that may be utilized by each of the first, second and third synchronization markers 465c, 465a, 465b, respectively, for the respective acquisitions of the bilateral link 114, the first link 112L and the second link 112R. The exemplary synchronization marker 465 preferably comprises unique pair ID field 703 m that may store the unique pair IDs which may comprise a unique number or code. A unique pair ID may comprise between 8 bits and 32 bits. A first unique pair ID may be utilized to pair the first hearing device 100L and the first hearing implant 150L to each other. The first unique pair ID may be stored in a memory of the master processing unit 120L and further stored in a memory of the first control unit 108L of the first hearing implant 150L. The first unique pair ID may for example be stored during manufacture of the first or master hearing device 100L or later in connection with a fitting of the binaural hearing system 125 to the user. The first unique pair ID may be stored in a similar manner in the memory of the first control unit 108L. A second unique pair ID may be utilized to pair the second hearing device 100R and the second hearing implant 150R to each other. The second unique pair ID may be stored in the memory of the second processing unit 120R and further stored in a memory of the second control unit 108R of the second hearing implant 150R. The second unique pair ID may for example be stored in the second processing unit 120R and the second control unit 108R in a similar manner to the first unique pair ID as outlined above.

A third unique pair ID may be utilized to pair the second hearing device 100R and the first hearing device 100L to each other. The third unique pair ID may be stored in the memory of the first processing unit 120L and further stored in the memory of the second processing unit 120R. The third unique pair ID may for example be stored in the second processing unit 120R and the first processing unit 120L in a similar manner to the first unique pair ID as outlined above.

The use of unique pair IDs like the first, second and third unique pair IDs, ensures that only the appropriate pair of devices are connected to each other during the respective acquisitions of the first link 112L, second link 112R and bilateral link 114. Otherwise, unintended crosstalk between the devices, for example cross-talk between the first and second hearing implants 150L, 150R of the binaural hearing system 125 could lead to erroneous pairing of devices. The first, second and third unique pair IDs may prevent in a similar manner unintended crosstalk and erroneous pairing of corresponding devices of two different but nearby located binaural hearing systems. The synchronization marker 465 can be viewed as special type of data packet and may have the same length and number of bits as at least some of the bilateral data packets. The synchronization marker 465 may comprise a predetermined search sequence 701 for example a predetermined binary pattern like 10101010 or equivalently 01010101. The latter is a priori known to both the master processing unit 120, the slave processing unit 120R and each of the first and second control units 108L, 108R for example by storage in respective memories of the units at manufacture. The predetermined binary pattern may be received and recognized by the slave processing unit 120R to detect the first synchronization marker 465c in a currently unoccupied time slot at the slave processing unit 120R. The predetermined binary pattern may likewise be received and recognized by the first control unit 108L of the first hearing implant 150L to detect the second synchronization marker 465a in a currently unoccupied time slot at the first control unit 108L. The predetermined binary pattern may likewise be received and recognized by the second control unit 108R of the second hearing implant 150R in a similar manner.

The synchronization marker 465 may comprise an optional wrap-around count-down number 707. The latter may be utilized by the master processing unit 120L and slave processing unit 120R for providing an additional layer of safety once the slave processing unit 120R and master processing unit 120L have acknowledged each other through the bilateral link 114 as described in additional detail below. A slot indicator 709 tells the slave processing unit 120R in which time slot at the master side the synchronization marker 465 is arranged. This feature allows the slave processing unit 120R to align its time slots with the corresponding time slots at the master processing unit 120L. The slave processing unit 120R may for example determine a time offset between already known time slots at the slave processing unit 120R and the corresponding time slots at the master processing unit 120L for alignment. Since, the length of a time slot, e.g. 48 µs, is known to the slave processing unit 120R at least via the common communication protocol the time offset can indicate slot alignment.

FIG. 8 illustrates an exemplary super-frame structure where each super-frame comprises a plurality of individual frames, such as between 4 and 32 individual frames, of the plurality of consecutive frames in accordance with one embodiment of the common communication protocol. The frames at the master side of the binaural hearing system are structured in consecutive first super-frames 901 and the super-frames at the slave side of the binaural hearing system are structured in consecutive second super-frames 951. Each frame such as frame-1 may carry the same type of data for example digital audio signals which means that all data packets of the frame in question hold the same type of data e.g. digital audio signals, control information or error-correction codes like CRC and/or forward error-correction codes (FEC) etc. The super-frames at the master and slave sides may be misaligned in time, i.e. a time off-set, as schematically illustrated by an off-set arrow 953. The slave processing unit 120R may be configured to detect this time off-set and transmit that to the master processing unit 120L which proceeds to align the first and second super-frames 901, 951 between the master side and slave side.

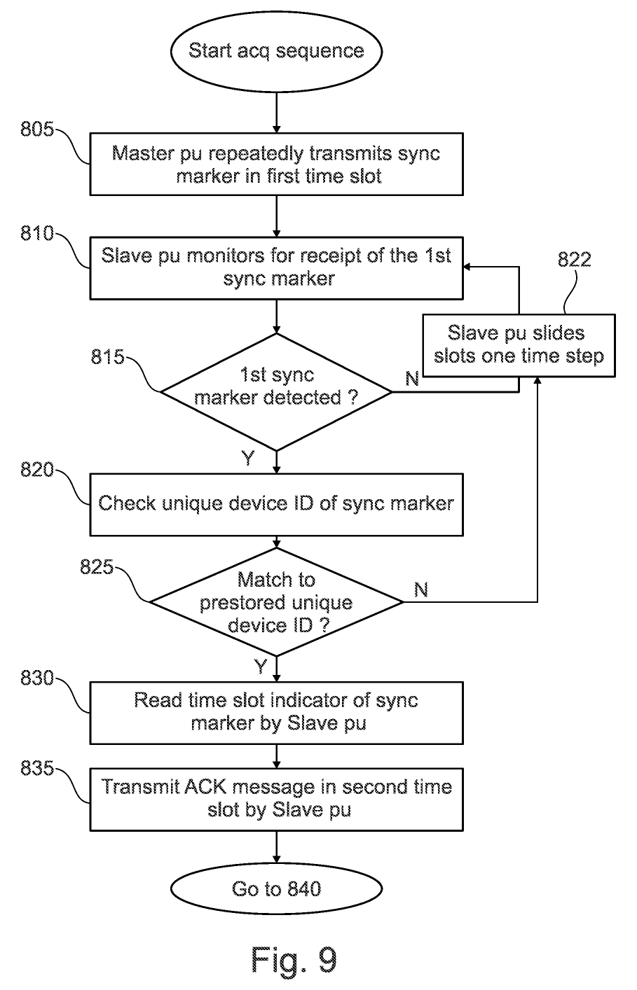

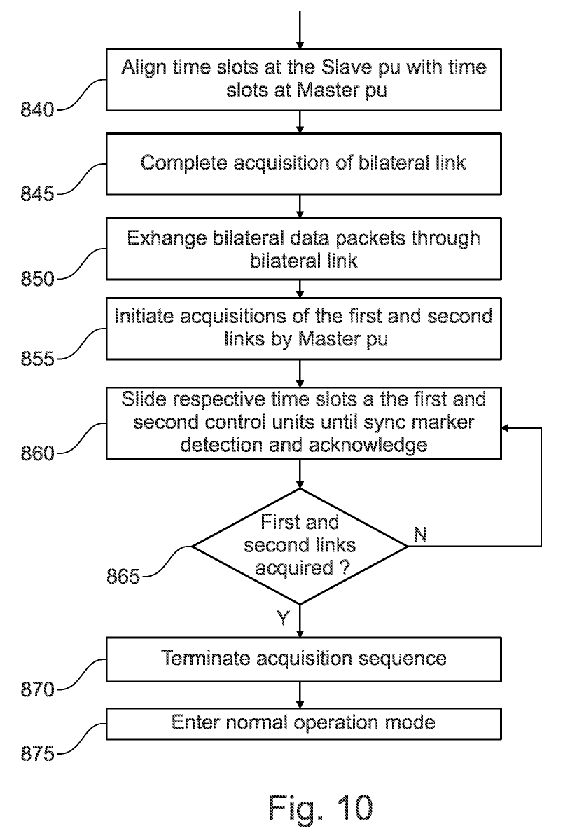

FIG. 9 and FIG. 10 are flow charts illustrating respective processing steps 805 – 875 carried out by the master and slave processing units 120L, 120R of the first and second hearing devices 100L, 100R in connection with the above-outlined acquisition sequence according to embodiments of the exemplary binaural hearing system 125.

### LIST OF REFERENCE SYMBOLS

100L first/master hearing device

100R second/slave hearing device

102L first magnetic coil antenna of the first hearing implant

102R second magnetic coil antenna of the second hearing implant

104L first rechargeable battery assembly

104R second rechargeable battery assembly

106L first electrode array

106R second electrode array

107L first power line/wire

107R second power line/wire

108L first control unit/ first implant processor

108R second control unit/ second implant processor

110L first receiver (Rx) charging coil

110R second receiver (Rx) charging coil

112L first bidirectional wireless communication link/ first link

112R second bidirectional wireless communication link/ second link

114 bilateral bidirectional wireless communication link/ bilateral link/ third link

116L first magnetic coil antenna of the first hearing device

116R second magnetic coil antenna of the second hearing device

118L first transceiver

118R second transceiver

120L first/master processing unit

120R second/slave processing unit

122L first data bus

122R second data bus

124L one or more first microphones

124R one or more second microphones

125 binaural hearing system

126L first system clock generator

126R second system clock generator

128L first radio interface

128R first radio interface (of second hearing device)

130L first RF antenna

130R second RF antenna

150L first hearing implant

150R second hearing implant

250 user’s head

405, 410 second ipsilateral data packets

430, 480 bilateral data packets

455, 460 first ipsilateral data packets

465 layout of synchronization marker

465a second synchronization marker

465b third synchronization marker

465c first synchronization marker

470 acknowledge message (ACK)

471 time slot

571 time slot

573 time slot

575 first time slide arrow

577 second time slide arrow

701 predetermined search sequence

703 unique pair ID field

707 countdown wrap-around counter

709 slot indicator

711 parity bit

805 – 875 processing steps of acquisition sequence

901 first super-frames

951 second super-frames

1. A binaural hearing system comprising:
a first hearing device, a second hearing device, a first hearing implant and a second hearing implant;
wherein the first hearing device is communicatively connectable to the first hearing implant via a first bidirectional wireless communication link for exchange of first ipsilateral data packets;
wherein the second hearing device is communicatively connectable to the second hearing implant via a second bidirectional wireless communication link for exchange of second ipsilateral data packets;
wherein the first hearing device and second hearing device are communicatively connectable via a bilateral bidirectional wireless communication link for exchange of bilateral data packets;
wherein the bilateral bidirectional wireless communication link, and the first and second bidirectional wireless communication links are associated with a common communication protocol which comprises a plurality of consecutive frames;
wherein the binaural hearing system is configured to:
acquire the bilateral bidirectional wireless communication link using at least a first processing unit of the first hearing device and a second processing unit of the second hearing device,
acquire the first bidirectional wireless communication link using at least the first processing unit of the first hearing device after the bilateral bidirectional wireless communication link is acquired, and
acquire the second bidirectional wireless communication link using at least the second processing unit of the second hearing device after the bilateral bidirectional wireless communication link is acquired.

2. The binaural hearing system according to claim 1, wherein the binaural hearing system is configured to acquire the bilateral bidirectional wireless communication link by:
transmitting a first synchronization marker from the first processing unit to the second processing unit;
detecting the first synchronization marker at the second processing unit;
synchronizing a plurality of time slots at the second processing unit with a plurality of time slots at the first processing unit based on the first synchronization marker; and
transmitting an acknowledge message from the second processing unit to the first processing unit.

3. The binaural hearing system according to claim 2, wherein the binaural hearing system is configured to acquire the first bidirectional wireless communication link by:
transmitting a second synchronization marker from the first processing unit to a first implant processor of the first hearing implant;
sliding a plurality of time slots at the first implant processor;
detecting the second synchronization marker at the first implant processor;
synchronizing the plurality of time slots at first implant processor with the plurality of time slots at the first processing unit based on the second synchronization marker; and
transmitting an acknowledge message from the first implant processor to the first processing unit.

4. The binaural hearing system according to claim 3, wherein the binaural hearing system is configured to acquire the second bidirectional wireless communication link by:
transmitting a third synchronization marker from the second processing unit to a second implant processor of the second hearing implant;
sliding a plurality of time slots at the second implant processor;
detecting the third synchronization marker at the second implant processor;
synchronizing the plurality of time slots at second implant processor with the plurality of time slots at the second processing unit based on the third synchronization marker; and
transmitting an acknowledge message from the second implant processor to the second processing unit.

5. The binaural hearing system according to claim 4, wherein:
the first synchronization marker comprises a first ID for pairing the first hearing device and the second hearing device;
the second synchronization marker comprises a second ID for pairing the first hearing device and first hearing implant; and
the third synchronization marker comprises a third ID for pairing the second hearing device and the second hearing implant.

6. The binaural hearing system according to claim 2, wherein the second processing unit is configured to:
compare a first ID of the first synchronization marker with a pre-stored ID;
ignore the first synchronization marker if the first ID does not match the pre-stored ID; and
transmit the acknowledge message if the pre-stored ID matches the first ID of the first synchronization marker.

7. The binaural hearing system according to claim 1, wherein the common communication protocol comprises a plurality of super frames, wherein the plurality of consecutive frames is a part of one of the super frames.

8. The binaural hearing system according to claim 2, wherein the binaural hearing system is configured to acquire the bilateral bidirectional wireless communication link also by sliding the plurality of time slots at the second processing unit based on predetermined time steps using a wrap-around scheme.

9. The binaural hearing system according to claim 8, wherein each of the predetermined time steps is less than 5 % of a time slot length.

10. The binaural hearing system according to claim 1, wherein the binaural hearing system is configured to maintain the bilateral bidirectional wireless communication link while acquiring the first bidirectional wireless communication link and/or while acquiring the second bidirectional wireless communication link.

11. The binaural hearing system according to claim 1, wherein the second processing unit is configured to determine an offset between super frames at the second processing unit, and provide the offset for transmission to the first processing unit via the bilateral bidirectional wireless communication link; and
wherein the binaural hearing system is configured to align super frames at the first processing unit with the super frames at the second processing unit.

12. The binaural hearing system according to according to claim 2, wherein the first synchronization marker comprises:
a predetermined binary pattern,
a time slot indicator indicating a certain time slot holding the synchronization marker,
an error-detecting code, or
any combination of two or more of the foregoing.

13. The binaural hearing system according to claim 1, wherein each frame of the plurality of consecutive frames comprises at least four non-overlapping time slots.

14. The binaural hearing system according to claim 13, wherein a length of each of the at least four non-overlapping time slots is identical.

15. The binaural hearing system according to claim 1, wherein at least a subset of the first and second ipsilateral data packets comprises digital audio data.

16. The binaural hearing system according to claim 1, wherein at least a subset of the bilateral data packets comprises digital audio data.

17. A method performed by a binaural hearing system comprising a first hearing device, a first hearing implant, a second hearing device, and a second hearing implant, the method comprising:
acquiring a bilateral bidirectional wireless communication link between the first hearing device and the second hearing device using a first processing unit of the first hearing device and a second processing unit of the second hearing device;
exchanging bilateral data packets between the first hearing device and the second hearing device;
acquiring a first bidirectional wireless communication link between the first hearing device and the first hearing implant using the first processing unit of the first hearing device, after the bilateral bidirectional wireless communication link is acquired;
exchanging first ipsilateral data packets between the first hearing device and the first hearing implant;
acquiring a second bidirectional wireless communication link between the second hearing device and second hearing implant using the second processing unit of the second hearing device, after the bilateral bidirectional wireless communication link is acquired;
exchanging second ipsilateral data packets between the second hearing device and the second hearing implant; and
entering a normal operation mode of the binaural hearing system.

18. The method according to claim 17, wherein:
the bilateral data packets are exchanged in first and second time slots; and
the first ipsilateral data packets are exchanged in third and fourth time slots.

19. The method according to claim 17, wherein act of acquiring the bilateral bidirectional wireless communication link comprises synchronizing a plurality of time slots at the second processing unit with a plurality of time slots at the first processing unit based on a first synchronization marker;
wherein the act of acquiring the first bidirectional wireless communication link comprises synchronizing a plurality of time slots at first implant processor with the plurality of time slots at the first processing unit based on a second synchronization marker; and
wherein the act of acquiring the second bidirectional wireless communication link comprises synchronizing a plurality of time slots at second implant processor with the plurality of time slots at the second processing unit based on a third synchronization marker.

20. The method according to claim 17, wherein the act of acquiring the first bidirectional wireless communication link comprises sliding a plurality of time slots at the first implant processor; and
wherein the act of acquiring the second bidirectional wireless communication link comprises sliding a plurality of time slots at the second implant processor.
